(19) United States
(12) Patent Application Publication
(10) Pub. No.: US 2021/0254013 A1
(43) Pub. Date: Aug. 19, 2021

(54) USE OF FIBROBLASTS AND/OR MODIFIED FIBROBLASTS FOR THREE DIMENSIONAL TISSUE PRINTING

(71) Applicant: Figene, LLC, Houston, TX (US)

(72) Inventors: Pete O'Heeron, Houston, TX (US); Thomas Ichim, San Diego, CA (US)

(21) Appl. No.: 17/251,587

(22) PCT Filed: Jun. 14, 2019
(86) PCT No.: PCT/US2019/037310

§ 371 Date: Dec. 11, 2020

Related U.S. Application Data
(60) Provisional application No. 62/684,844, filed on Jun. 14, 2018.

(51) Int. Cl. C12N 5/071; C12N 5/00; B33Y 10/00; B33Y 80/00; B33Y 70/00

(52) U.S. Cl. CPC C12N 5/0671; C12N 5/0062; B33Y 10/00; B33Y 80/00; B33Y 70/00; C12N 2513/00; C12N 2501/999; C12N 2533/90; C12N 2533/50; C12N 2502/1323; C12N 2500/02

(57) ABSTRACT

Embodiments of the disclosure encompass methods and compositions related to bioprinting processes that utilize fibroblasts in a structural and/or functional capacity. In specific embodiments, fibroblasts or cells derived therefrom are utilized in a bioprinting process. Cells derived from fibroblasts include cells that derive from fibroblasts that were dedifferentiated and then re-differentiated into cells of a desired phenotype for use in the bioprinting process. Such bioprinting processes may produce specific tissues, organoids and/or organs.

This application claims priority to U.S. Provisional Patent Application Ser. No. 62/684,844, filed Jun. 14, 2018, which is incorporated by reference herein in its entirety.

### TECHNICAL FIELD

The disclosure pertains to the field of cell biology and tissue engineering, more particularly, the subject matter of the disclosure pertains to generation of three dimensional organs, organoids, and/or tissues. The disclosure pertains to field of fibroblasts as cells for use in three dimensional bioprinting.

### BACKGROUND

The science of tissue engineering is based on using cells, supporting material—scaffolds, growth factors and in many cases bioreactors, to grow tissue and organs. A major factor pushing for the development of artificial or in vitro-generated organs has been a shortage of organs that are needed for transplantation. Tremendous scientific and technological progress has been made in the past 20 years that has made it possible to grow almost all human tissues and many organs. In recent years, the pharmaceutical and cosmetic industry has shown great interest in applying advances in tissue engineering to grow tissue and “mini” organs for drug discovery and drug testing. The new regulations are making restrictions for using animals for testing of cosmetic products. This has initiated tremendous interest for developing human skin models, as in “skin on the plate”. For the generation of organs, organoids, and/or tissues, it is important to generate a three-dimensional (3D) environment similar to a native tissue environment to be able to migrate, proliferate, and/or differentiate to develop functional tissues. Likewise, regenerative cells typically need a 3D environment to differentiate into desired cell lineage. This is the reason why scaffolds with 3D architecture and specific microporosity have been developed for tissue engineering applications. In classical tissue engineering studies, cells are seeded in a 3D scaffold and then cultivated in an incubator or stimulated in a bioreactor or directly implanted in vivo.

Although numerous types of stem cells have been utilized for the purpose of three dimensional printing, there is still a major need for a reliable source of cells that is easy to procure, can be obtained in large numbers, and does not require excessive or cumbersome ex vivo expansion. The present disclosure satisfies such a need.

### BRIEF SUMMARY

Embodiments of the disclosure include methods and compositions for the generation of one or more tissues, one or more organoids, and/or one or more organs. In particular embodiments, fibroblasts are utilized in the generation of one or more tissues, one or more organoids, and/or one or more organs, including at least as a starting cell in a process for producing the one or more tissues, one or more organoids, and/or one or more organs, although in other cases the fibroblasts are utilized to produce a composition (for example, one or more scaffolds or one or more matrices) that are then utilized with another type of cell (and/or fibroblasts).

Disclosed herein include means of using fibroblasts cells in the generation of organs and/or organoid tissues using three dimensional (3D) printing. In one embodiment, fibroblasts are utilized as a substitute for stem cells of any kind, including at least mesenchymal stem cells. In another embodiment, fibroblasts are used for generation of scaffolding means, which are subsequently used to seed cells of other tissue types. In another embodiment, fibroblasts are de-differentiated and used to seed de-cellularized organ matrices in order to generate ex vivo organs.

In cases in which a particular type of tissue, organoid, and/or organ is desired, the cells for the appropriate tissue, organoid, and/or organ may be of any kind. However, in at least specific cases the cells for the desired tissue, organoid, and/or organ are cells that are dedifferentiated fibroblasts, meaning that fibroblasts were manipulated to dedifferentiate into certain cells and then those cells are then subject to suitable conditions to differentiate into the desired cells for the tissue, organoid, and/or organ. In alternative cases, fibroblasts are manipulated to dedifferentiate into the desired cells for the tissue, organoid, and/or organ. The dedifferentiation of the fibroblasts may occur by any suitable method, such as upon culture with cytoplasm from cells that are more undifferentiated as compared to standard fibroblasts (such cells may be stem cells or any kind, for example). In addition to, or as an alternative, fibroblasts may be dedifferentiated upon exposure to hypoxia, in the presence of one or more HDAC inhibitors, upon exposure to one or more DNA methyltransferase inhibitors, and/or upon culture with one or more factors, including particular growth factors. Hypoxia levels include oxygen at hypoxic conditions comprise an oxygen range between 0.2%-5%, 0.2%-4%, 0.2%-3%, 0.2%-2%, 0.2%-1%, 0.2-0.5%, 0.5%-5%, 0.5%-4%, 0.5%-3%, 0.5%-2%, 0.5%-1%, 1%-5%, 1%-4%, 1%-3%, 1%-2%, 2%-5%, 2%-4%, 2%-3%, 3%-5%, 3%-4%, 4%-5%, and so on.

In some cases, fibroblasts (or dedifferentiated fibroblasts) are utilized for the process of bioprinting in lieu of utilizing one or more types of stem cells, although in particular cases fibroblasts or dedifferentiated fibroblasts are utilized in conjunction with stem cells in bioprinting.

In embodiments of the disclosure, there is a bio-ink comprising fibroblasts and/or dedifferentiated fibroblasts. In specific embodiments, the bio-ink comprises fibroblasts and/or dedifferentiated fibroblasts, and a suitable extracellular matrix or mixtures of suitable extracellular matrices. The bio-ink may comprise suitable nutrients, growth factors, amino acids, electrolytes, hormones, growth factors, etc. The bio-ink may be suitably formulated for storage and/or transport. Fibroblasts and/or dedifferentiated fibroblasts may be autologous with respect to an individual or allogeneic with respect to an individual.

Particular embodiments encompass methods of producing tissue, organoids and/or organs by exposing fibroblasts and/or dedifferentiated fibroblasts to a polymeric or hydrogel matrix (in some embodiments, said matrix may comprise extracellular matrix) to produce at least one layer or matrix and cells, particularly in a predetermined pattern. Upon subsequent layering of the cells and matrix, the fibroblasts and/or dedifferentiated fibroblasts become embedded in the matrix and under suitable conditions they fuse to form a desired 3D tissue organoid and/or organ. Upon using standard modeling practices for bioprinting that dictate the structural evolution of the cells with the matrix, patterns are utilized to achieve suitable organ printing processes to produce the desired 3D structure.

Embodiments of the disclosure encompass methods of generating an organ, organoid, and/or tissue, comprising the steps of a. culturing fibroblasts under suitable conditions to induce dedifferentiation of fibroblasts; and b. using said dedifferentiated fibroblasts, or differentiated cells produced therefrom, in a bio-printing process to produce the organ, organoid and/or tissue. The fibroblasts may be selected from the group consisting of dermal fibroblasts, placental fibroblasts, adipose fibroblasts, bone marrow fibroblasts, foreskin fibroblasts, umbilical cord fibroblasts, hair follicle-derived fibroblasts, nail-derived fibroblasts, endometrial derived fibroblasts, keloid derived fibroblasts, and a combination thereof. In specific embodiments, in the culturing step the fibroblasts are subjected to hypoxic conditions, such as that comprise a reduced oxygen range between 0.2%-5%. The culturing step may comprise culturing of the fibroblasts in a culture medium treated with one or more epigenetic modifiers, such as one or more of the following: DNA demethylating agent, histone deacetylase inhibitor, histone modifier, or a combination thereof. The DNA demethylating agent may be selected from the group consisting of 5-azacytidine, N-methyl-N′-nitro-N-nitrosoguanidine (MNNG), Temozolomide, Procarbazine, and a combination thereof. The histone deacetylase inhibitor may be selected from the group consisting of Valproic acid, Trichostatin A, Phenylbutyrate, Vorinostat, Belinostat, LAQ824, Panobinostat, Entinostat, CI994, Mocetinostat, Sulforaphane, and a combination thereof. The histone modifier may be selected from the group consisting of poly ADP ribose polymerase, Enhancer of zeste homolog 2 (EZH2), valproic acid, trichostatine, and a combination thereof.

In some cases, the fibroblasts are transfected with cytoplasm derived from stem cells, including stem cells selected from the group consisting of parthenogenic stem cells, embryonic stem cells, inducible pluripotent stem cells, somatic cell nuclear transfer derived stem cells, and a combination thereof. In some cases, fibroblasts transfected with cytoplasm derived from pluripotent stem cells become dedifferentiated. In specific embodiments, dedifferentiated fibroblasts express detectable levels of one or more genes selected from the group consisting of alkaline phosphatase (ALP), OCT4, SOX2, human telomerase reverse transcriptase (hERT), SSEA-4, NANOG, and a combination thereof. The dedifferentiated fibroblasts may be cultured using a culture medium treated with one or more growth factors, such as growth factors selected from the group consisting of FGF-1, FGF-2, FGF-5, EGF, CNTF, KGF-1, PDGF, platelet-rich plasma, TGF-alpha, HGF-1, and a combination thereof.

In particular embodiments, dedifferentiated fibroblasts, or differentiated cells produced therefrom, are cultured as a plurality of cell aggregates. The fibroblasts, dedifferentiated fibroblasts, or differentiated cells produced therefrom, may be cultured as a plurality of cell aggregates prior to the bioprinting process. The cell aggregates may be seeded in extracellular matrix, such as extracellular matrix selected from the group consisting of mammalian extracellular matrix, piscine extracellular matrix, plant extracellular matrix, and a combination thereof. In certain embodiments, the method further comprises the steps of generating extracellular matrix from fibroblast lysates. Cell aggregates may be seeded in extracellular matrix that is configured into the form of a defined shape. Cell aggregates seeded in extracellular matrix may be introduced into a mold having the defined shape.

In any embodiments of the disclosure fibroblasts, dedifferentiated fibroblasts, or differentiated cells produced therefrom are subjected to hypoxic conditions. In specific cases, the extracellular matrix is cultured using a culture medium treated with one or more cell attachment peptides (such as RGD peptide), one or more cell attachment proteins, one or more cytokines, one or more glycosaminoglycans, or a combination thereof. In cases wherein cytokines are employed, the cytokine may be selected from the group consisting of vascular endothelial growth factor (VEGF), bone morphogenetic protein (BMP), adrenomedullin (AM), angiopoietin (Ang), brain-derived neurotrophic factor (BDNF), epidermal growth factor (EGF), erythropoietin (Epo), fibroblast growth factor (FGF), glial cell line-derived neurotrophic factor (GNDF), granulocyte colony stimulating factor (G-CSF), granulocyte-macrophage colony stimulating factor (GM-CSF), growth differentiation factor (GDF-9), hepatocyte growth factor (HGF), hepatoma-derived growth factor (HDGF), insulin-like growth factor (IGF), migration-stimulating factor, myostatin (GDF-8), myelomonocytic growth factor (MGF), nerve growth factor (NGF), placental growth factor (P1GF), platelet-derived growth factor (PDGF), thrombopoietin (Tpo), transforming growth factor alpha (TGFα), transforming growth factor beta (TGFβ), tumor necrosis factor alpha (TNFα), Wnt protein, and a combination thereof. The glycosaminoglycan is selected from the group consisting of hyaluronate, chondroitin sulfate, heparin sulfate, heparin, dermatan sulfate, and keratin sulfate, and a combination thereof.

Some methods further compris admixing a hydrogel during deposition of the matrix onto said cells or cellular aggregates. Certain methods further comprise deposition of a synthetic polymer onto the cells and/or cellular aggregates alone, or that have been admixed with a hydrogel. A synthetic polymer may be selected from the group consisting of poly (L-lactide-co-glycolide), poly lactic-co-glycolic acid (PLGA), Polycaprolactone (PLC), Polylactic acid, Polybutylene terephthalate, Polyethylene terephthalate, Polyethylene glycol, and a combination thereof. In specific cases, dedifferentiated fibroblasts and/or differentiated cells produced therefrom are of an endodermal, ectodermal, or mesodermal lineage. The dedifferentiated fibroblasts may be differentiated into cells of a desired type.

Dedifferentiated fibroblasts may be differentiated into cells of a desired type selected from the group consisting of salivary gland mucous cells, salivary gland serous cells, von Ebner's gland cells, mammary gland cells, lacrimal gland cells, ceruminous gland cells, eccrine sweat gland dark cells, eccrine sweat gland clear cells, apocrine sweat gland cells, gland of Moll cells, sebaceous gland cells, Bowman's gland cells, Brunner's gland cells, seminal vesicle cells, prostate gland cells, bulbourethral gland cells, Bartholin's gland cells, Littre gland cells, uterus endometrium cells, goblet cells, stomach lining mucous cells, gastric gland zymogenic cells, gastric gland oxyntic cells, pancreatic acinar cells, paneth cells, type II pneumocytes, clara cells, somatotropes, lactotropes, thyrotropes, gonadotropes, corticotropes, intermediate pituitary cells, magnocellular neurosecretory cells, gut cells, respiratory tract cells, thyroid epithelial cells, parafollicular cells, parathyroid gland cells, parathyroid chief cells, oxyphil cells, adrenal gland cells, chromaffin cells, Leydig cells, theca interna cells, corpus luteum cells, granulosa lutein cells, theca lutein cells, juxtaglomerular cells, macula densa cells, peripolar cells, mesangial cells, blood vessel and lymphatic vascular endothelial fenestrated cells, blood vessel and lymphatic vascular endothelial continuous cells, blood vessel and lymphatic vascular endothelial splenic cells, synovial cells, peritoneal serosal cells, pleural serosal cells, pericardial cavity serosal cells, squamous cells, columnar cells, dark cells, vestibular membrane cells, stria vascularis basal cells, stria vascularis marginal cells, cells of Claudius, cells of Boettcher, choroid plexus cells, arachnoid squamous cells, pigmented ciliary epithelium cells, non-pigmented ciliary epithelium cells, corneal endothelial cells, peg cells, respiratory tract ciliated cells, oviduct ciliated cells, uterine endometrial ciliated cells, rete testis ciliated cells, ductulus efferens ciliated cells, ciliated ependymal cells, epidermal keratinocytes, epidermal basal cells, fingernail and toenail keratinocytes, nail bed basal cells, medullary hair shaft cells, cortical hair shaft cells, cuticular hair shaft cells, cuticular hair root sheath cells, hair root sheath cells of Huxley's layer, hair root sheath cells of Henle's layer, external hair root sheath cells, hair matrix cells, stratified squamous epithelium, epithelial basal cells, urinary epithelium cells, inner auditory hair cells of the organ of Corti, outer auditory hair cells of the organ of Corti, basal cells of olfactory epithelium, cold-sensitive primary sensory neurons, heat-sensitive primary sensory neurons, epidermal Merkel cells, olfactory receptor neurons, pain-sensitive primary sensory neurons, photoreceptor rod cells, photoreceptor blue-sensitive cone cells, photoreceptor green-sensitive cone cells, photoreceptor red-sensitive cone cells, proprioceptive primary sensory neurons, touch-sensitive primary sensory neurons, type I carotid body cells, type II carotid body cells, type I hair cell of the vestibular apparatus of the ear, type II hair cell of the vestibular apparatus of the ear, type I taste bud cells, cholinergic neural cells, adrenergic neural cells, peptidergic neural cells, inner pillar cells of the organ of Corti, outer pillar cells of the organ of Corti, inner phalangeal cells of the organ of Corti, outer phalangeal cells of the organ of Corti, border cells of the organ of Corti, Hensen cells of the organ of Corti, vestibular apparatus supporting cells, taste bud supporting cells, olfactory epithelium supporting cells, Schwann cells, satellite cells, enteric glial cells, astrocytes, neurons, oligodendrocytes, spindle neurons, anterior lens epithelial cells, crystallin-containing lens fiber cells, hepatocytes, adipocytes, white fat cells, brown fat cells, liver lipocytes, kidney glomerulus parietal cells, kidney glomerulus podocytes, kidney proximal tubule brush border cells, loop of Henle thin segment cells, kidney distal tube cells, kidney collecting duct cells, type I pneumocytes, pancreatic duct cells, non-striated duct cells, duct cells, intestinal brush border cells, exocrine gland striated duct cells, gall bladder epithelial cells, ductus efferens non-ciliated cells, epididymal principal cells, epididymal basal cells, ameloblast epithelial cells, planum semilunatum epithelial cells, organ of Corti interdental epithelial cells, loose connective tissue fibroblasts, corneal keratocytes, tendon fibroblasts, bone marrow reticular tissue fibroblasts, non-epithelial fibroblasts, pericytes, nucleus pulposus cells, cementoblast/cementocytes, odontoblasts, odontocytes, hyaline cartilage chondrocytes, fibrocartilage chondrocytes, elastic cartilage chondrocytes, osteoblasts, osteocytes, osteoclasts, osteoprogenitor cells, hyalocytes, cochlear stellate cells, hepatic stellate cells, pancreatic stellate cells, red skeletal muscle cells, white skeletal muscle cells, intermediate skeletal muscle cells, nuclear bag cells of the muscle spindle, nuclear chain cells of the muscle spindle, satellite cells, cardiomyocytes, nodal cardiomyocytes, Purkinje fiber cells, smooth muscle cells, myoepithelial cells of the iris, myoepithelial cells of the exocrine glands, reticulocytes, megakaryocytes, monocytes, connective tissue macrophages, epidermal Langerhans cells, dendritic cells, microglial cells, neutrophils, eosinophils, basophils, mast cells, helper T cells, suppressor T cells, cytotoxic T cells, natural killer T cells, B cells, natural killer cells, melanocytes, retinal pigmented epithelial cells, oogonia/oocytes, spermatids, spermatocytes, spermatogonium cells, spermatozoa, ovarian follicle cells, Sertoli cells, thymus epithelial cells, intestinal kidney cells, and a mixture thereof.

In specific embodiments, bioprinting comprises three-dimensional printing of a biological organ, organoid, and/or tissue through the layering of living cells using a bioprinter. The bioprinter is a three-axis mechanical platform that controls the movements of extruders that deposit layers of living cells in a desired shape. In specific cases, the desired shape is acquired by scanning the surface of a desired organ, organoid and/or tissue to generate a surface map for guidance with cell deposition. In at least some cases, scanning the surface of a desired organ, organoid and/or tissue is achieved using a laser, electron beam, magnetic resonance imaging, microwave, x-ray, computed tomography, or a combination thereof.

Embodiments of the disclosure encompass a method of preparing cells for a bioprinting process, comprising the step of exposing fibroblasts to suitable conditions to cause dedifferentiation of the fibroblasts. The dedifferentiated fibroblasts may be subject to suitable conditions to differentiate into differentiated cells of a desired type, such as cells selected from the group consisting of salivary gland mucous cells, salivary gland serous cells, von Ebner's gland cells, mammary gland cells, lacrimal gland cells, ceruminous gland cells, eccrine sweat gland dark cells, eccrine sweat gland clear cells, apocrine sweat gland cells, gland of Moll cells, sebaceous gland cells, Bowman's gland cells, Brunner's gland cells, seminal vesicle cells, prostate gland cells, bulbourethral gland cells, Bartholin's gland cells, Littre gland cells, uterus endometrium cells, goblet cells, stomach lining mucous cells, gastric gland zymogenic cells, gastric gland oxyntic cells, pancreatic acinar cells, paneth cells, type II pneumocytes, clara cells, somatotropes, lactotropes, thyrotropes, gonadotropes, corticotropes, intermediate pituitary cells, magnocellular neurosecretory cells, gut cells, respiratory tract cells, thyroid epithelial cells, parafollicular cells, parathyroid gland cells, parathyroid chief cells, oxyphil cells, adrenal gland cells, chromaffin cells, Leydig cells, theca interna cells, corpus luteum cells, granulosa lutein cells, theca lutein cells, juxtaglomerular cells, macula densa cells, peripolar cells, mesangial cells, blood vessel and lymphatic vascular endothelial fenestrated cells, blood vessel and lymphatic vascular endothelial continuous cells, blood vessel and lymphatic vascular endothelial splenic cells, synovial cells, peritoneal serosal cells, pleural serosal cells, pericardial cavity serosal cells, squamous cells, columnar cells, dark cells, vestibular membrane cells, stria vascularis basal cells, stria vascularis marginal cells, cells of Claudius, cells of Boettcher, choroid plexus cells, arachnoid squamous cells, pigmented ciliary epithelium cells, non-pigmented ciliary epithelium cells, corneal endothelial cells, peg cells, respiratory tract ciliated cells, oviduct ciliated cells, uterine endometrial ciliated cells, rete testis ciliated cells, ductulus efferens ciliated cells, ciliated ependymal cells, epidermal keratinocytes, epidermal basal cells, fingernail and toenail keratinocytes, nail bed basal cells, medullary hair shaft cells, cortical hair shaft cells, cuticular hair shaft cells, cuticular hair root sheath cells, hair root sheath cells of Huxley's layer, hair root sheath cells of Henle's layer, external hair root sheath cells, hair matrix cells, stratified squamous epithelium, epithelial basal cells, urinary epithelium cells, inner auditory hair cells of the organ of Corti, outer auditory hair cells of the organ of Corti, basal cells of olfactory epithelium, cold-sensitive primary sensory neurons, heat-sensitive primary sensory neurons, epidermal Merkel cells, olfactory receptor neurons, pain-sensitive primary sensory neurons, photoreceptor rod cells, photoreceptor blue-sensitive cone cells, photoreceptor green-sensitive cone cells, photoreceptor red-sensitive cone cells, proprioceptive primary sensory neurons, touch-sensitive primary sensory neurons, type I carotid body cells, type II carotid body cells, type I hair cell of the vestibular apparatus of the ear, type II hair cell of the vestibular apparatus of the ear, type I taste bud cells, cholinergic neural cells, adrenergic neural cells, peptidergic neural cells, inner pillar cells of the organ of Corti, outer pillar cells of the organ of Corti, inner phalangeal cells of the organ of Corti, outer phalangeal cells of the organ of Corti, border cells of the organ of Corti, Hensen cells of the organ of Corti, vestibular apparatus supporting cells, taste bud supporting cells, olfactory epithelium supporting cells, Schwann cells, satellite cells, enteric glial cells, astrocytes, neurons, oligodendrocytes, spindle neurons, anterior lens epithelial cells, crystallin-containing lens fiber cells, hepatocytes, adipocytes, white fat cells, brown fat cells, liver lipocytes, kidney glomerulus parietal cells, kidney glomerulus podocytes, kidney proximal tubule brush border cells, loop of Henle thin segment cells, kidney distal tube cells, kidney collecting duct cells, type I pneumocytes, pancreatic duct cells, non-striated duct cells, duct cells, intestinal brush border cells, exocrine gland striated duct cells, gall bladder epithelial cells, ductus efferens non-ciliated cells, epididymal principal cells, epididymal basal cells, ameloblast epithelial cells, planum semilunatum epithelial cells, organ of Corti interdental epithelial cells, loose connective tissue fibroblasts, corneal keratocytes, tendon fibroblasts, bone marrow reticular tissue fibroblasts, non-epithelial fibroblasts, pericytes, nucleus pulposus cells, cementoblast/cementocytes, odontoblasts, odontocytes, hyaline cartilage chondrocytes, fibrocartilage chondrocytes, elastic cartilage chondrocytes, osteoblasts, osteocytes, osteoclasts, osteoprogenitor cells, hyalocytes, cochlear stellate cells, hepatic stellate cells, pancreatic stellate cells, red skeletal muscle cells, white skeletal muscle cells, intermediate skeletal muscle cells, nuclear bag cells of the muscle spindle, nuclear chain cells of the muscle spindle, satellite cells, cardiomyocytes, nodal cardiomyocytes, Purkinje fiber cells, smooth muscle cells, myoepithelial cells of the iris, myoepithelial cells of the exocrine glands, reticulocytes, megakaryocytes, monocytes, connective tissue macrophages, epidermal Langerhans cells, dendritic cells, microglial cells, neutrophils, eosinophils, basophils, mast cells, helper T cells, suppressor T cells, cytotoxic T cells, natural killer T cells, B cells, natural killer cells, melanocytes, retinal pigmented epithelial cells, oogonia/oocytes, spermatids, spermatocytes, spermatogonium cells, spermatozoa, ovarian follicle cells, Sertoli cells, thymus epithelial cells, and intestinal kidney cells. In certain embodiments, methods comprise the step of employing the differentiated cells of a desired type in a bioprinting process.

The foregoing has outlined rather broadly the features and technical advantages of the present disclosure in order that the detailed description that follows may be better understood. Additional features and advantages will be described hereinafter which form the subject of the claims herein. It should be appreciated by those skilled in the art that the conception and specific embodiments disclosed may be readily utilized as a basis for modifying or designing other structures for carrying out the same purposes of the present designs. It should also be realized by those skilled in the art that such equivalent constructions do not depart from the spirit and scope as set forth in the appended claims. The novel features which are believed to be characteristic of the designs disclosed herein, both as to the organization and method of operation, together with further objects and advantages will be better understood from the following description when considered in connection with the accompanying FIGURES. It is to be expressly understood, however, that each of the FIGURES is provided for the purpose of illustration and description only and is not intended as a definition of the limits of the present disclosure.

### BRIEF DESCRIPTION OF THE DRAWING

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawing (also “FIGURE” and “FIG.” herein), of which:

FIG. 1 shows enhanced viability in co-culture with fibroblasts as a function of time. Bars on the left are HepG2 alone; bars in the middle are 1:2 fibroblasts to HepG2; bars on the right are 1:1 fibroblasts to HepG2 Cells.

While various embodiments of the disclosure have been shown and described herein, it will be obvious to those skilled in the art that such embodiments are provided by way of example only. Numerous variations, changes, and substitutions may occur to those skilled in the art without departing from the disclosure. It should be understood that various alternatives to the embodiments of the disclosure described herein may be employed.

### DETAILED DESCRIPTION

### I. Examples of Definitions

As used herein, “allograft” refers to an organ or tissue derived from a genetically non-identical member of the same species as the recipient.

As used herein, “bio-ink” refers to a liquid, semi-solid, or solid composition comprising a plurality of cells. In some embodiments, bio-ink comprises cell solutions, cell aggregates, cell-comprising gels, multicellular bodies, and/or tissues. In some embodiments, the bio-ink additionally comprises support materials, said support materials may include a member of the collagen family, aggrecan, syndecan, or other polymeric agents. In some embodiments, the bio-ink additionally comprises non-cellular materials that provide specific biomechanical properties that enable bioprinting.

As used herein, “bioprinting” refers to utilizing three-dimensional, precise deposition of cells (e.g., cell solutions, cell-containing gels, cell suspensions, cell concentrations, multicellular aggregates, multicellular bodies, combinations thereof, etc.) via methodology that is compatible with an automated, computer-aided, three-dimensional prototyping device (e.g., a bioprinter).

As used herein, “cartridge” refers to any object that is capable of receiving (and holding) a bio-ink or a support material.

As used herein, a “computer module” refers to a software component (including a section of code) that interacts with a larger computer system. In some embodiments, a software module (or program module) comes in the form of a file and typically handles a specific task within a larger software system. In some embodiments, a module may be included in one or more software systems. In other embodiments, a module may be seamlessly integrated with one or more other modules into one or more software systems. A computer module is optionally a stand-alone section of code or, optionally, code that is not separately identifiable. A key feature of a computer module is that it allows an end user to use a computer to perform the identified functions.

As used herein, term “engineered,” when used to refer to tissues and/or organs, refers to that cells, cell solutions, cell suspensions, cell-comprising gels, cell-comprising pastes, cell concentrates, multicellular aggregates, and/or layers and/or mixtures thereof are positioned to form three-dimensional structure(s) by a computer-aided device (e.g., a bioprinter), for example, according to computer code. In further embodiments, the computer script is, for example, one or more computer programs, computer applications, and/or computer modules. In still further embodiments, three-dimensional tissue structures form through the post-printing fusion of cells or multicellular bodies similar to self-assembly phenomena in early morphogenesis.

As used herein, “implantable” refers to biocompatible and capable of being inserted or grafted into or affixed onto a living organism either temporarily or substantially permanently.

As used herein, “organ” refers to a collection of tissues joined into structural unit to serve a common function. Examples of organs include, but are not limited to, skin, sweat glands, sebaceous glands, mammary glands, bone, brain, hypothalamus, pituitary gland, pineal body, heart, blood vessels, larynx, trachea, bronchus, lung, lymphatic vessel, salivary glands, mucous glands, esophagus, stomach, gallbladder, liver, pancreas, small intestine, large intestine, colon, urethra, kidney, adrenal gland, conduit, ureter, bladder, fallopian tube, uterus, ovaries, testes, prostate, thyroid, parathyroid, meibomian gland, parotid gland, tonsil, adenoid, thymus, and spleen.

As used herein, “patient” refers to any individual. The term is interchangeable with “subject,” “recipient,” “individual,” and “donor.” None of the terms should be construed as requiring the supervision (constant or otherwise) of a medical professional (e.g., physician, nurse, nurse practitioner, physician's assistant, orderly, hospice worker, social worker, clinical research associate, etc.) or a scientific researcher, although they may be involved. The patient may be a mammal, including a human, dog, cat, horse, pig, sheep, and so forth.

As used herein, “tissue” refers to an aggregate of cells. Examples of tissues include, but are not limited to, connective tissue (e.g., areolar connective tissue, dense connective tissue, elastic tissue, reticular connective tissue, and/or adipose tissue), muscle tissue (e.g., skeletal muscle, smooth muscle and/or cardiac muscle), genitourinary tissue, gastrointestinal tissue, pulmonary tissue, bone tissue, nervous tissue, and epithelial tissue (e.g., simple epithelium and/or stratified epithelium), hematopoietic tissue, endoderm-derived tissue, mesoderm-derived tissue, and/or ectoderm-derived tissue.

As used herein, “xenograft” refers to an organ and/or tissue derived from a different species as the recipient.

### II. General Embodiments

Embodiments of the disclosure concern the use of fibroblasts and/or dedifferentiated fibroblasts for use in three dimensional bioprinting of tissues, organs, and/or organoids. Fibroblasts may be utilized in a variety of ways, including as a starting substrate for cells to be utilized in the generation of tissues, organoids, and/or organs. Such fibroblasts may be manipulated under suitable conditions to become dedifferentiated cells that are subsequently differentiated into cells other than fibroblasts; the newly differentiated cells are then utilized in bioprinting processes to produce the tissue, organoid, and/or organ.

The disclosure encompasses bioprinting of various cells, depending on a desired outcome. In one embodiment the disclosure concerns the use of fibroblasts as a replacement or substitute for other types of cells (such as stem cells, including at least mesenchymal stem cells) for the process of bioprinting. In one embodiment, the disclosure concerns the utilization of dedifferentiated fibroblasts as a replacement or substitute for stem cells for the process of bioprinting. In another embodiment, the disclosure provides for the use of dedifferentiated fibroblasts as a means of starting cells for generation therefrom of other differentiated cells needed in the process of 3D bioprinting. In the practice of some methods of the disclosure, various bio-ink compositions may be used, said bio-ink compositions usually comprising of extracellular matrix to obtain a multi-dimensional construct having a pre-determined pattern, wherein the bio-ink composition comprises one or a plurality of extracellular matrices. Additionally, the disclosure provides means capable of transporting nutrients, blood supplies, growth factors, amino acids, electrolytes, gases, hormones, blood cells and/or other organic materials to the in vitro growing 3D organ. In a method of organ printing, a plurality of cell aggregates are embedded in a polymeric or gel matrix and allowed to fuse to form a desired three-dimensional tissue structure. An intermediate product may comprise at least one layer of matrix and a plurality of cell aggregates embedded therein, for example in a predetermined pattern. Modeling methods may be utilized to predict the structural evolution of fusing cell aggregates for combinations of cell type, matrix, and embedding patterns to enable selection of organ printing processes parameters for use in producing an engineered tissue having a desired three-dimensional structure.

### III. Fibroblasts in Bioprinting and Methods Thereof

In some embodiments, devices, systems, and methods for fabricating tissues and organs are utilized as means of assembling 3D biological structures in which fibroblasts are utilized as structural and/or functional materials. In some embodiments, the devices are bioprinters. In some embodiments, the methods comprise the use of bioprinting techniques. In further embodiments, the tissues and organs fabricated by use of the devices, systems, and methods described herein are bioprinted. In some embodiments, bioprinted cellular constructs, tissues, and/or organs are generated with a method that utilizes a rapid prototyping technology based on three-dimensional, automated, computer-aided deposition of cells, including cell solutions, cell suspensions, cell-comprising gels or pastes, cell concentrations, multicellular bodies (e.g., cylinders, spheroids, ribbons, etc.), and/or support material onto a biocompatible surface (e.g., composed of hydrogel and/or a porous membrane) by a three-dimensional delivery device (e.g., a bioprinter). A number of methods are available to arrange cells, multicellular aggregates, and/or layers thereof on a biocompatible surface to produce a three-dimensional structure, such methods including manual placement or positioning by an automated, computer-aided machine such as a bioprinter. Advantages of delivery of cells or multicellular bodies with bioprinting technology include rapid, accurate, and reproducible placement of cells or multicellular bodies to produce constructs exhibiting planned or pre-determined orientations or patterns of cells, multicellular aggregates and/or layers thereof with various compositions. Advantages also include assured high cell density while minimizing cell damage.

In some embodiments, methods of bioprinting are continuous and/or substantially continuous. A non-limiting example of a continuous bioprinting method is to dispense bio-ink from a bioprinter via a dispense tip (e.g., a syringe, capillary tube, etc.) connected to a reservoir of bio-ink. In further non-limiting embodiments, a continuous bioprinting method is to dispense bio-ink in a repeating pattern of functional units. In various embodiments, a repeating functional unit has any suitable geometry, including, for example, circles, squares, rectangles, triangles, polygons, irregular geometries, or a combination thereof. In further embodiments, a repeating pattern of bioprinted functional units comprises at least one layer, and a plurality of layers are bioprinted adjacently (e.g., stacked) to form an engineered tissue, organoid, and/or organ. In various embodiments, 2, 3, 4, 5, 6, 7, 8, 9, 10, 11, 12, 13, 14, 15, or more layers are bioprinted adjacently (e.g., stacked) to form an engineered tissue or organ.

In some embodiments, fibroblasts or undifferentiated fibroblasts, may be used as part of the composition of the bio-ink. In some embodiments, bio-ink comprises liquid or semi-solid cell solutions, cell suspensions, or cell concentrations. In further embodiments, a cell solution, suspension, or concentration comprises a liquid or semi-solid (e.g., viscous) carrier and a plurality of cells. In still further embodiments, the carrier is a suitable cell nutrient media, such as those described herein. In some embodiments, bio-ink comprises semi-solid or solid multicellular aggregates or multicellular bodies. In further embodiments, the bio-ink is produced by 1) mixing a plurality of cells or cell aggregates and a biocompatible liquid or gel in a pre-determined ratio to result in bio-ink; and 2) compacting the bio-ink to produce a bio-ink with a desired cell density and viscosity. In some embodiments, the compacting of the bio-ink is achieved by centrifugation, tangential flow filtration (“TFF”), or a combination thereof. In some embodiments, the compacting of the bio-ink results in a composition that is extrudable, allowing formation of multicellular aggregates or multicellular bodies. In some embodiments, “extrudable” refers to the ability to be shaped by forcing (e.g., under pressure) through a nozzle or orifice (e.g., one or more holes or tubes). In some embodiments, the compacting of the bio-ink results from growing the cells to a suitable density. The cell density necessary for the bio-ink will vary with the cells being used and the tissue or organ being produced. In some embodiments, the cells of the bio-ink are cohered and/or adhered. In some embodiments, “cohere,” “cohered,” and “cohesion” refer to cell-cell adhesion properties that bind cells, multicellular aggregates, multicellular bodies, and/or layers thereof. In further embodiments, the terms are used interchangeably with “fuse,” “fused,” and “fusion.” In some embodiments, the bio-ink additionally comprises support material, cell culture medium, extracellular matrix (or components thereof), cell adhesion agents, cell death inhibitors, anti-apoptotic agents, anti-oxidants, extrusion compounds, and combinations thereof. In various embodiments, there are bio-inks that include liquid, semi-solid, or solid compositions comprising a plurality of cells. In some embodiments, bio-ink comprises liquid or semi-solid cell solutions, cell suspensions, or cell concentrations. In some embodiments, any mammalian cell is suitable for use in bio-ink and in the fabrication of tissues and organs using the devices, systems, and methods described herein. In various embodiments, the cells are any suitable cell. In further various embodiments, the cells are vertebrate cells, mammalian cells, human cells, or combinations thereof. The cell may be differentiated from a fibroblast, in some cases. In some embodiments, the type of cell used in a method disclosed herein depends on the type of cellular construct, tissue, or organ being produced. In some embodiments, the bio-ink comprises one type of cell (also referred to as a “homologous ink”). In some embodiments, the bio-ink comprises more than one type of cell (also referred to as a “heterologous ink”).

In one embodiment of the disclosure, fibroblast or other cells for production of organs, organoids, and/or tissues may be obtained from cadaveric sources and organoids or organs may be decellularized. Means of decellularizing tissue including physical, chemical, and biochemical methods. See, e.g. U.S. Pat. No. 5,192,312 which is incorporated herein by reference. Such methods may be employed in accordance with the process(es) described herein. However, the decellularization technique employed in some cases does not result in gross disruption of the anatomy of the placental tissue or substantially alter the biomechanical properties of its structural elements, and in at least some cases leaves the placental vasculature substantially intact. In certain embodiments, the treatment of the placental tissue to produce a decellularized tissue matrix does not leave a cytotoxic environment that mitigates against subsequent repopulation of the matrix with cells that are allogeneic or autologous to the recipient. As used herein, cells and tissues that are “allogeneic” to the recipient are those that originate with or are derived from a donor of the same species as a recipient of the placental vascular scaffold, and “autologous” cells or tissues are those that originate with or are derived from a recipient of the placental vascular scaffold. The decellularized tissues may be subsequently seeded with various cells that are grown in tissue culture. In one embodiment, the cells are fibroblast cells. In other embodiments, fibroblasts are dedifferentiated prior to addition of the decellularized tissues.

In one embodiment of the disclosure, bioprinting is performed on placental extracellular matrix, wherein the placental extracellular matrix comprises base-treated and/or detergent treated Type I telopeptide placental collagen that has not been chemically modified or contacted with a protease, wherein the extracellular matrix comprises less than 5% fibronectin or less than 5% laminin by weight; between 25% and 92% Type I collagen by weight; and 2% to 50% Type III collagen or 2% to 50% type IV collagen by weight. In a further embodiment, the placental extracellular matrix comprises base-treated, detergent treated Type I telopeptide placental collagen that has not been chemically modified or contacted with a protease, wherein the extracellular matrix comprises less than 1% fibronectin or less than 1% laminin by weight; between 74% and 92% Type I collagen by weight; and 4% to 6% Type III collagen or 2% to 15% type IV collagen by weight.

Growth of the printed composition maybe performed using systems that are perfusing organs such as Transmedics, “Organ Care System”, Organ Recovery Systems “LifePort” technologies and the Toronto XVIVO Lung Perfusion System. In this system, an “ex vivo” or outside the body technique is utilized capable of continuously perfusing or pumping a bloodless solution containing oxygen, proteins and nutrients into injured donor lungs. This technique allows the surgeons the opportunity to assess and treat injured donor lungs, while they are outside the body, to make them suitable for transplantation.

In some cases, fibroblasts are placed onto, or adjacent to in a planar manner, an extracellular matrix, followed by placement of an additional layer of extracellular matrix on top of the fibroblasts or on the other side of the extracellular matrix, again in a planar manner. Such a process of alternating steps of placement of fibroblasts and placement of extracellular matrix may continue until a desired thickness and/or shape, or end result of any kind, is achieved. In specific embodiments, the extracellular matrix is utilized with a hydrogel, including in a particular ratio in some cases. Examples of ratios include extracellular matrix to hydrogel in a range of 15:1 to 1:15 by weight. In specific cases, the ratio of extracellular matrix to hydrogel is or is about 15:1, 14:1, 13:1, 12:1, 11:1, 10:1, 9:1, 8:1, 7:1, 6:1, 5:1, 4:1, 3:1, 2:1, or 1:1. In certain cases, the ratio of extracellular matrix to hydrogel is or is about 1:15, 1:14, 1:13, 1:12, 1:11, 1:10, 1:9, 1:8, 1:7, 1:6, 1:5, 1:4, 1:3, or 1:2. In particular cases the methods for generating the structure of the tissue, organoid, and/or organ includes the deposition of a synthetic polymer, said deposition being made in the tissue, organoid, or organ that is being assembled.

Embodiments of the disclosure include methods of generating a three dimensional organ, organoid, and/or tissue comprising the steps of: a) obtaining at least one cell type; b) placing said cell type(s) onto an extracellular matrix; c) placing an additional layer of matrix above said cell type; and d) repeating the process of steps “b” and “c” in a manner to form said desired organ, organoid, and/or tissue. The cells and/or extracellular matrix may be deposited onto a solid surface of any kind, and in specific cases the solid surface is comprised of a biocompatible material. The cells and extracellular matrix may or may not be printed onto each other. In specific cases, the cells and extracellular matrix are printed onto each other on top of a surface. In certain cases, the method comprises scanning (for example, by use of a laser, electron beam, magnetic resonance imaging, microwave, x-ray, and/or computed tomography) a surface of an organ, organoid, and/or tissue, whose production is desired so as to form a surface map, and using the surface map to guide the depositing. In specific embodiments, the extracellular matrix is selected from the group consisting of a) mammalian extracellular matrix (for example, generated from fibroblast lysates); b) molluscan mammalian extracellular matrix; c) piscene extracellular matrix; d) plant extracellular matrix; and e) a combination thereof. The mammalian extracellular matrix may be adipose tissue-derived, Wharton's jelly derived, bone marrow-derived, or placentally-derived. In some cases, the methods further comprise deposition of a hydrogel into said tissue, organoid, or organ being assembled, including when the extracellular matrix and the hydrogel are combined in a ratio of about 15:1 to 1:15 by weight. In some cases, the method comprises deposition of a synthetic polymer, including a synthetic polymer selected from a group comprising of: a) poly(L-lactide-co-glycolide) (PLGA); b) polycaprolactone; c) polylactic acid; d) polybutylene terephthalate; e) polyethylene terephthalate; and f) polyethylene glycol, for example.

In some cases, fibroblasts are placed onto, or adjacent to in a planar manner, an extracellular matrix, followed by placement of an additional layer of extracellular matrix on top of the fibroblasts or on the other side of the extracellular matrix, again in a planar manner. Such a process of alternating steps of placement of fibroblasts and placement of extracellular matrix may continue until a desired thickness and/or shape, or end result of any kind, is achieved. In specific embodiments, the extracellular matrix is utilized with a hydrogel, including in a particular ratio in some cases. Examples of ratios include extracellular matrix to hydrogel in a range of 15:1 to 1:15 by weight. In specific cases, the ratio of extracellular matrix to hydrogel is or is about 15:1, 14:1, 13:1, 12:1, 11:1, 10:1, 9:1, 8:1, 7:1, 6:1, 5:1, 4:1, 3:1, 2:1, or 1:1. In certain cases, the ratio of extracellular matrix to hydrogel is or is about 1:15, 1:14, 1:13, 1:12, 1:11, 1:10, 1:9, 1:8, 1:7, 1:6, 1:5, 1:4, 1:3, or 1:2. In particular cases the methods for generating the structure of the tissue, organoid, and/or organ includes the deposition of a synthetic polymer, into the tissue, organoid, or organ being assembled.

In specific embodiments, an extracellular matrix comprises and/or is derivatized with one or more cell attachment peptides, one or more cell attachment proteins, one or more cytokines, and/or one or more glycosaminoglycans, for example. An example of a cytokine is selected from the group consisting of a) vascular endothelial growth factor (VEGF); b) bone morphogenetic protein (BMP); c) adrenomedullin (AM); d) angiopoietin (Ang); e) brain-derived neurotrophic factor (BDNF); f) epidermal growth factor (EGF); g) erythropoietin (Epo); h) fibroblast growth factor (FGF); i) glial cell line-derived neurotrophic factor (GNDF); j) granulocyte colony stimulating factor (G-CSF); k) granulocyte-macrophage colony stimulating factor (GM-CSF); l) growth differentiation factor (GDF-9); m) hepatocyte growth factor (HGF); n) hepatoma derived growth factor (HDGF); o) insulin-like growth factor (IGF); p) migration-stimulating factor; q) myostatin (GDF-8); r) myelomonocytic growth factor (MGF); s) nerve growth factor (NGF); t) placental growth factor (P1GF); u) platelet-derived growth factor (PDGF); v) thrombopoietin (Tpo); w) transforming growth factor alpha (TGF-.alpha.); x) TGF-.beta; y) tumor necrosis factor alpha (TNF-alpha), z) Wnt protein, and/or a′) a combination thereof. Cell attachment peptides may include a cell responsive peptide comprising one or more RGD motifs, for example.

In specific embodiments, the methods of generating a three-dimensional organ, organoid, and/or tissue utilize manipulation of a cell type(s) to be used with the extracellular matrix. In particular embodiments, the cell type used in generating the three-dimensional organ, organoid, and/or tissue is a fibroblast of any kind, including those obtained from a biopsy. In such cases, the donor providing the biopsy is either the patient to be treated (autologous) or the donor is different from the patient to be treated (allogeneic). The fibroblasts may be obtained from a source selected from the group consisting of a) dermal fibroblasts; b) placental fibroblasts; c) adipose fibroblasts; d) bone marrow fibroblasts; e) foreskin fibroblasts; f) umbilical cord fibroblasts; g) hair follicle derived fibroblasts; h) nail derived fibroblasts; i) endometrial derived fibroblasts; j) keloid derived fibroblasts; and k) a mixture thereof. The fibroblasts may be dedifferentiated, and induction of dedifferentiation of the fibroblasts may be performed by culture of the fibroblasts together with cytoplasm from a cell possessing a more undifferentiated phenotype, as compared to original fibroblasts. The cell possessing the more undifferentiated phenotype may be any type of stem cell, including a pluripotent stem cell, for example. In specific cases, the pluripotent stem cell is selected from the group of cells consisting of: a) parthenogenic stem cells; b) embryonic stem cells; c) inducible pluripotent stem cells; d) somatic cell nuclear transfer derived stem cells; e) Stimulus-triggered acquisition of pluripotency (STAP); and (f) a combination thereof.

In cases wherein there is induction of dedifferentiation of the fibroblasts by culturing of the fibroblasts together with cytoplasm from a cell that possesses a more undifferentiated phenotype, the culture of the fibroblasts with the undifferentiated cells may be performed under particular conditions, such as hypoxia. In such cases, the culture under hypoxia comprises of culture in conditions of reduced oxygen as compared to atmospheric oxygen. As an example, reduced oxygen may be between 0.2%-5% oxygen. In some cases, the culture is performed in the presence of one or more histone deacetylase inhibitors, and the fibroblast may be exposed to the histone deacetylase inhibitor at a concentration and frequency sufficient to enhance ability of the fibroblast to be useful for three-dimensional printing. In specific embodiments, the histone deacetylase inhibitor is selected from the group consisting of: a) valproic acid; b) trichostatin A; c) valproic acid; d) phenylbutyrate; e) vorinostat; f) belinostat; g) LAQ824; h) panobinostat; i) entinostat; j) CI994; k) mocetinostat; l) sulforaphane; and m) a combination thereof. The fibroblasts may be cultured in the presence of a DNA methyltransferase inhibitor at a concentration and frequency sufficient to enhance ability of said fibroblast to be useful for three-dimensional printing. The DNA methyltransferase inhibitor(s) may be selected from the group consisting of: a) decitabine; b) 5-azacytidine; c) Zebularine; d) RG-108; e) procaine hydrochloride; f) Procainamide hydrochloride; g) Hydralazine hydrochloride; h) Epigallocatechin gallate; i) Chlorogenic acid; j) Caffeic acid; k) a combination thereof.

The fibroblasts may or may not be dedifferentiated by transfection with one or more dedifferentiation agents, including, for example, cytoplasms derived from pluripotent stem cells. The cytoplasm derived from the pluripotent stem cells may be transfected into fibroblasts. Fibroblasts may be cultured in a media allowing for proliferation of fibroblasts in an undifferentiated state. Dedifferentiated fibroblasts may be grown in the presence of one or more factors selected from the group consisting of: a) FGF-1; b) FGF-2; c) FGF-5; d) EGF; e) CNTF; f) KGF-1; g) PDGF; h) platelet rich plasma; i) TGF-alpha; j) HGF-1; and k) a combination thereof. Dedifferentiated fibroblasts of any kind may be cultured under hypoxia.

In particular embodiments, cell types used for generation of the organ, organoid, and/or tissue may be mixtures of cells representing the desired organ, organoid, and/or tissue. The cell types may be fibroblasts that have been dedifferentiated. The dedifferentiated fibroblasts may be differentiated into cells of the desired phenotype or type for generation of organ, organoid, and/or tissue, and differentiated cells may be of the endodermal, ectodermal, or mesodermal lineages. In specific embodiments, the dedifferentiated fibroblasts may be differentiated into cells that are selected from the group of cells consisting of salivary gland mucous cells, salivary gland serous cells, von Ebner's gland cells, mammary gland cells, lacrimal gland cells, ceruminous gland cells, eccrine sweat gland dark cells, eccrine sweat gland clear cells, apocrine sweat gland cells, gland of Moll cells, sebaceous gland cells. bowman's gland cells, Brunner's gland cells, seminal vesicle cells, prostate gland cells, bulbourethral gland cells, Bartholin's gland cells, gland of Littre cells, uterus endometrium cells, isolated goblet cells, stomach lining mucous cells, gastric gland zymogenic cells, gastric gland oxyntic cells, pancreatic acinar cells, paneth cells, type II pneumocytes, clara cells, somatotropes, lactotropes, thyrotropes, gonadotropes, corticotropes, intermediate pituitary cells, magnocellular neurosecretory cells, gut cells, respiratory tract cells, thyroid epithelial cells, parafollicular cells, parathyroid gland cells, parathyroid chief cell, oxyphil cell, adrenal gland cells, chromaffin cells, Leydig cells, theca interna cells, corpus luteum cells, granulosa lutein cells, theca lutein cells, juxtaglomerular cell, macula densa cells, peripolar cells, mesangial cell, blood vessel and lymphatic vascular endothelial fenestrated cells, blood vessel and lymphatic vascular endothelial continuous cells, blood vessel and lymphatic vascular endothelial splenic cells, synovial cells, serosal cell (lining peritoneal, pleural, and pericardial cavities), squamous cells, columnar cells, dark cells, vestibular membrane cell (lining endolymphatic space of ear), stria vascularis basal cells, stria vascularis marginal cell (lining endolymphatic space of ear), cells of Claudius, cells of Boettcher, choroid plexus cells, pia-arachnoid squamous cells, pigmented ciliary epithelium cells, nonpigmented ciliary epithelium cells, corneal endothelial cells, peg cells, respiratory tract ciliated cells, oviduct ciliated cell, uterine endometrial ciliated cells, rete testis ciliated cells, ductulus efferens ciliated cells, ciliated ependymal cells, epidermal keratinocytes, epidermal basal cells, keratinocyte of fingernails and toenails, nail bed basal cells, medullary hair shaft cells, cortical hair shaft cells, cuticular hair shaft cells, cuticular hair root sheath cells, hair root sheath cells of Huxley's layer, hair root sheath cells of Henle's layer, external hair root sheath cells, hair matrix cells, surface epithelial cells of stratified squamous epithelium, basal cell of epithelia, urinary epithelium cells, auditory inner hair cells of organ of Corti, auditory outer hair cells of organ of Corti, basal cells of olfactory epithelium, cold-sensitive primary sensory neurons, heat-sensitive primary sensory neurons, Merkel cells of epidermis, olfactory receptor neurons, pain-sensitive primary sensory neurons, photoreceptor rod cells, photoreceptor blue-sensitive cone cells, photoreceptor green-sensitive cone cells, photoreceptor red-sensitive cone cells, proprioceptive primary sensory neurons, touch-sensitive primary sensory neurons, type I carotid body cells, type II carotid body cell (blood pH sensor), type I hair cell of vestibular apparatus of ear (acceleration and gravity), type II hair cells of vestibular apparatus of ear, type I taste bud cells cholinergic neural cells, adrenergic neural cells, peptidergic neural cells, inner pillar cells of organ of Corti, outer pillar cells of organ of Corti, inner phalangeal cells of organ of Corti, outer phalangeal cells of organ of Corti, border cells of organ of Corti, Hensen cells of organ of Corti, vestibular apparatus supporting cells, taste bud supporting cells, olfactory epithelium supporting cells, Schwann cells, satellite cells, enteric glial cells, astrocytes, neurons, oligodendrocytes, spindle neurons, anterior lens epithelial cells, crystallin-containing lens fiber cells, hepatocytes, adipocytes, white fat cells, brown fat cells, liver lipocytes, kidney glomerulus parietal cells, kidney glomerulus podocytes, kidney proximal tubule brush border cells, loop of Henle thin segment cells, kidney distal tubule cells, kidney collecting duct cells, type I pneumocytes, pancreatic duct cells, nonstriated duct cells, duct cells, intestinal brush border cells, exocrine gland striated duct cells, gall bladder epithelial cells, ductulus efferens nonciliated cells, epididymal principal cells, epididymal basal cells, ameloblast epithelial cells, planum semilunatum epithelial cells, organ of Corti interdental epithelial cells, loose connective tissue fibroblasts, corneal keratocytes, tendon fibroblasts, bone marrow reticular tissue fibroblasts, nonepithelial fibroblasts, pericytes, nucleus pulposus cells, cementoblast/cementocytes, odontoblasts, odontocytes, hyaline cartilage chondrocytes, fibrocartilage chondrocytes, elastic cartilage chondrocytes, osteoblasts, osteocytes, osteoclasts, osteoprogenitor cells, hyalocytes, stellate cells (ear), hepatic stellate cells (Ito cells), pancreatic stelle cells, red skeletal muscle cells, white skeletal muscle cells, intermediate skeletal muscle cells, nuclear bag cells of muscle spindle, nuclear chain cells of muscle spindle, satellite cells, ordinary heart muscle cells, nodal heart muscle cells, Purkinje fiber cells, smooth muscle cells, myoepithelial cells of iris, myoepithelial cell of exocrine glands, reticulocytes, megakaryocytes, monocytes, connective tissue macrophages. epidermal Langerhans cells, dendritic cells, microglial cells, neutrophils, eosinophils, basophils, mast cells, helper T cells, suppressor T cells, cytotoxic T cells, natural Killer T cells, B cells, natural killer cells, melanocytes, retinal pigmented epithelial cells, oogonia/oocytes, spermatids, spermatocytes, spermatogonium cells, spermatozoa, ovarian follicle cells, Sertoli cells, thymus epithelial cell, and/or interstitial kidney cells.

### IV. Manipulation of Fibroblasts for Bioprinting Methods

Embodiments of the disclosure encompass methods of generating one or more organs, one or more organoids, and/or one or more tissues by utilizing fibroblasts in a 3D bioprinting process. The process may utilize standard bioprinting steps but utilizes fibroblasts, or cells produced therefrom, as the cells component of a bioprinting process. In specific embodiments, the methods for generating one or more organs, one or more organoids, and/or one or more tissues incorporates the manipulation of fibroblasts to become dedifferentiated, followed by the utilization of the dedifferentiated fibroblasts, or re-differentiated cells derived from the dedifferentiated fibroblasts, in the bioprinting process.

Generation of differentiated fibroblasts for use in the methods of the disclosure may be performed to endow augmentation of plasticity, thus allowing for increased efficacy in generation of cells useful for therapeutic means. In particular embodiments, the disclosure provides fibroblasts with augmented plasticity so as to increase therapeutic success. By using epigenetic modifications and/or culture in appropriate conditions (at least in some cases), the present methods can allow for dedifferentiation or transdifferentiate cells of a recipient, e.g., an individual in need of cell or gene therapy (autologous), or of a donor (allogeneic). In the case of autologous use, the methods of the disclosure solve the problem of immunorejection as cells from one patient can be transformed into a different type of cell, thereby allowing for the production or creation of specific types of cells needed for the treatment of a particular disease the patient may be suffering from. For example, pancreatic islet cells for the treatment of diabetes or hepatocytes for the treatment of liver disease, or chondrocytes for treatment of joints and cartilage regeneration may be produced. Also, this disclosure provides for the formation of donor dedifferentiated cells, such as pluripotent cells, e.g., stem cells thereby allowing for the derivation of different somatic cell phenotypes therefrom. In addition, while the cells produced according to the disclosure are especially desired for cell therapy they may also be used for study of mechanisms involved in cell differentiation and disease progression. Dedifferentiated cells can either remain dedifferentiated and proliferate as a dedifferentiated cell; redifferentiate along the same developmental pathway from which the cell had previously dedifferentiated; or redifferentiate along a developmental pathway distinct from which the cell had previously dedifferentiated. Within the context of the present disclosure, a dedifferentiated fibroblast possesses enhanced plasticity and ability to differentiate, or “redifferentiate” into other cells, including at least chondrocytes, notochord, salivary gland mucous cells, salivary gland serous cells, von Ebner's gland cells, mammary gland cells, lacrimal gland cells, ceruminous gland cells, eccrine sweat gland dark cells, eccrine sweat gland clear cells, apocrine sweat gland cells, gland of Moll cells, sebaceous gland cells. bowman's gland cells, Brunner's gland cells, seminal vesicle cells, prostate gland cells, bulbourethral gland cells, Bartholin's gland cells, gland of Littre cells, uterus endometrium cells, isolated goblet cells, stomach lining mucous cells, gastric gland zymogenic cells, gastric gland oxyntic cells, pancreatic acinar cells, paneth cells, type II pneumocytes, clara cells, somatotropes, lactotropes, thyrotropes, gonadotropes, corticotropes, intermediate pituitary cells, magnocellular neurosecretory cells, gut cells, respiratory tract cells, thyroid epithelial cells, parafollicular cells, parathyroid gland cells, parathyroid chief cell, oxyphil cell, adrenal gland cells, chromaffin cells, Leydig cells, theca interna cells, corpus luteum cells, granulosa lutein cells, theca lutein cells, juxtaglomerular cell, macula densa cells, peripolar cells, mesangial cell, blood vessel and lymphatic vascular endothelial fenestrated cells, blood vessel and lymphatic vascular endothelial continuous cells, blood vessel and lymphatic vascular endothelial splenic cells, synovial cells, serosal cell (lining peritoneal, pleural, and pericardial cavities), squamous cells, columnar cells, dark cells, vestibular membrane cell (lining endolymphatic space of ear), stria vascularis basal cells, stria vascularis marginal cell (lining endolymphatic space of ear), cells of Claudius, cells of Boettcher, choroid plexus cells, pia-arachnoid squamous cells, pigmented ciliary epithelium cells, nonpigmented ciliary epithelium cells, corneal endothelial cells, peg cells, respiratory tract ciliated cells, oviduct ciliated cell, uterine endometrial ciliated cells, rete testis ciliated cells, ductulus efferens ciliated cells, ciliated ependymal cells, epidermal keratinocytes, epidermal basal cells, keratinocyte of fingernails and toenails, nail bed basal cells, medullary hair shaft cells, cortical hair shaft cells, cuticular hair shaft cells, cuticular hair root sheath cells, hair root sheath cells of Huxley's layer, hair root sheath cells of Henle's layer, external hair root sheath cells, hair matrix cells, surface epithelial cells of stratified squamous epithelium, basal cell of epithelia, urinary epithelium cells, auditory inner hair cells of organ of Corti, auditory outer hair cells of organ of Corti, basal cells of olfactory epithelium, cold-sensitive primary sensory neurons, heat-sensitive primary sensory neurons, Merkel cells of epidermis, olfactory receptor neurons, pain-sensitive primary sensory neurons, photoreceptor rod cells, photoreceptor blue-sensitive cone cells, photoreceptor green-sensitive cone cells, photoreceptor red-sensitive cone cells, proprioceptive primary sensory neurons, touch-sensitive primary sensory neurons, type I carotid body cells, type II carotid body cell (blood pH sensor), type I hair cell of vestibular apparatus of ear (acceleration and gravity), type II hair cells of vestibular apparatus of ear, type I taste bud cells cholinergic neural cells, adrenergic neural cells, peptidergic neural cells, inner pillar cells of organ of Corti, outer pillar cells of organ of Corti, inner phalangeal cells of organ of Corti, outer phalangeal cells of organ of Corti, border cells of organ of Corti, Hensen cells of organ of Corti, vestibular apparatus supporting cells, taste bud supporting cells, olfactory epithelium supporting cells, Schwann cells, satellite cells, enteric glial cells, astrocytes, neurons, oligodendrocytes, spindle neurons, anterior lens epithelial cells, crystallin-containing lens fiber cells, hepatocytes, adipocytes, white fat cells, brown fat cells, liver lipocytes, kidney glomerulus parietal cells, kidney glomerulus podocytes, kidney proximal tubule brush border cells, loop of Henle thin segment cells, kidney distal tubule cells, kidney collecting duct cells, type I pneumocytes, pancreatic duct cells, nonstriated duct cells, duct cells, intestinal brush border cells, exocrine gland striated duct cells, gall bladder epithelial cells, ductulus efferens nonciliated cells, epididymal principal cells, epididymal basal cells, ameloblast epithelial cells, planum semilunatum epithelial cells, organ of Corti interdental epithelial cells, loose connective tissue fibroblasts, corneal keratocytes, tendon fibroblasts, bone marrow reticular tissue fibroblasts, nonepithelial fibroblasts, pericytes, nucleus pulposus cells, cementoblast/cementocytes, odontoblasts, odontocytes, hyaline cartilage chondrocytes, fibrocartilage chondrocytes, elastic cartilage chondrocytes, osteoblasts, osteocytes, osteoclasts, osteoprogenitor cells, hyalocytes, stellate cells (ear), hepatic stellate cells (Ito cells), pancreatic stelle cells, red skeletal muscle cells, white skeletal muscle cells, intermediate skeletal muscle cells, nuclear bag cells of muscle spindle, nuclear chain cells of muscle spindle, satellite cells, ordinary heart muscle cells, nodal heart muscle cells, Purkinje fiber cells, smooth muscle cells, myoepithelial cells of iris, myoepithelial cell of exocrine glands, reticulocytes, megakaryocytes, monocytes, connective tissue macrophages, epidermal Langerhans cells, dendritic cells, microglial cells, neutrophils, eosinophils, basophils, mast cells, helper T cells, suppressor T cells, cytotoxic T cell, natural Killer T cells, B cells, natural killer cells, melanocytes, retinal pigmented epithelial cells, oogonia/oocytes, spermatids, spermatocytes, spermatogonium cells, spermatozoa, ovarian follicle cells, Sertoli cells, thymus epithelial cells, and/or interstitial kidney cells.

The state of a treated fibroblast cell that becomes dedifferentiated but is subsequently differentiated can be verified by histology and/or by increased or decreased expression of one or more genes that are characteristic(s) of the cell. The expression of one or more genes may be used as a marker(s) of the undifferentiated state. Genes associated with the undifferentiated state may be selected from the group consisting of alkaline phosphatase (ALP), OCT4, SOX2, human telomerase reverse transcriptase (hERT) and SSEA-4. To achieve dedifferentiation (induction into an undifferentiated state), the somatic cells are introduced with one or more reprogramming gene, such as OCT-4, Nanog, or SOX-2 in order to induce a dedifferentiation process. Said dedifferentiation process is quantified by alkaline phosphatase staining (AP staining), and furthermore, expression of Oct4 is verified by immunofluorescence (IF), for example using an Oct4 antibody. Finally, the MET degree in the dedifferentiation process of the somatic cells is verified by flow cytometry (FACS) using antibodies of THY1 as a marker of human dermal fibroblasts and an epithelial cell adhesion molecule (EPCAM) as a marker of the epithelial cell.

In one embodiment, dedifferentiation of fibroblasts is accomplished by (1) exposure to one or more epigenetic modifiers, such as one or more DNA demethylating agents, one or more HDAC inhibitors, one or more histone modifiers; and/or (2) exposure to one or more cell cycle manipulation agents and/or one or more pluripotent-promoting agents; and/or one or more tissue specific-promoting agents (such agents include helper cells that promote growth of pluripotent cells, growth factors, hormones, and bioactive molecules, for example). Examples of DNA methylating agents include at least 5-azacytidine (5-aza), MNNG, 5-aza, N-methl-N′-nitro-N-nitrosoguanidine, temozolomide, procarbazine, etc. Examples of methylation inhibiting drugs agents include at least decitabine, 5-azacytidine, hydralazine, procainamide, mitoxantrone, zebularine, 5-fluorodeoxycytidine, 5-fluorocytidine, anti-sense oligonucleotides against DNA methyltransferase, or other inhibitors of enzymes involved in the methylation of DNA. Examples of histone deacetylase (“HDAC”) inhibitors include hydroxamic acids, cyclic peptides, benzamides, short-chain fatty acids, and/or depudecin. Examples of hydroxamic acids and derivatives of hydroxamic acids include, but are not limited to, trichostatin A (TSA), suberoylanilide hydroxamic acid (SAHA), oxamflatin, suberic bishydroxamic acid (SBHA), m-carboxycinnamic acid bishydroxamic (CBHA), and pyroxamide. Examples of cyclic peptides include, but are not limited to, trapoxin A, apicidin and FR901228. Examples of benzamides include but are not limited to MS-27-275. Examples of short-chain fatty acids include but are not limited to butyrates (e.g., butyric acid and phenylbutyrate (PB)) Other examples include CI-994 (acetyldinaline) and trichostatine. Particular examples of histone modifiers include PARP, the human enhancer of zeste, valproic acid, and trichostatine. Particular constituents in an example of a media in order to facilitate RNA transformation and dedifferentiation of RNA-comprising target cells into pluripotent cells include trichostatine, valproic acid, zebularine and/or 5-aza.

In a particular embodiment, fibroblasts are treated with a variety of dedifferentiated compositions that can endow increased pluripotency. In one aspect of the disclosure, fibroblasts are treated with cytoplasm from a more undifferentiated cell. Such cells, including pluripotent stem cells, are well known in the art and methods of derivation are published and incorporated by reference. Without limitation, useful pluripotent cells of extraction of cytoplasm include parthenogenic stem cells [1-15], embryonic stem cells [16, 17], inducible pluripotent stem cells [18-22], Stimulus-triggered acquisition of pluripotency (STAP) [23], and/or somatic cell nuclear transfer derived stem cells [24-26].

Extraction of cytoplasmic matter may be performed as described in the art. In one embodiment, pluripotent cells are made to enter the interphase stage of cell cycle and are harvested using standard methods and washed by centrifugation at 500×g for 10 minutes in a 10 ml conical tube at 4° C. The supernatant is discarded, and the cell pellet is resuspended in a total volume of 50 ml of cold PBS. The cells are centrifuged at 500×g for 10 minutes at 4.degree. C. This washing step is repeated, and the cell pellet is resuspended in approximately 20 volumes of ice-cold interphase cell lysis buffer (20 mM Hepes, pH 8.2, 5 mM MgCl.sub.2, 1 mM DTT, 10 .mu.M aprotinin, 10 .mu.M leupeptin, 10 .mu.M pepstatin A, 10 .mu.M soybean trypsin inhibitor, 100 .mu.M PMSF, and preferably 20 .mu.g/ml cytochalasin B). The cells are sedimented by centrifugation at 800.times.g for 10 minutes at 4.degree. C. The supernatant is discarded, and the cell pellet is carefully resuspended in no more than one volume of interphase cell lysis buffer. The cells are incubated on ice for one hour to allow swelling of the cells. The cells are lysed by either sonication using a tip sonicator or Dounce homogenization using a glass mortar and pestle. Cell lysis is performed until at least 90% of the cells and nuclei are lysed, which may be assessed using phase contrast microscopy. The sonication time required to lyse at least 90% of the cells and nuclei may vary depending on the type of cell used to prepare the extract. The cell lysate is placed in a 1.5-ml centrifuge tube and centrifuged at 10,000 to 15,000×g for 15 minutes at 4° C. using a table top centrifuge. The tubes are removed from the centrifuge and immediately placed on ice. The supernatant is carefully collected using a 200 μl pipette tip, and the supernatant from several tubes is pooled and placed on ice. This supernatant is the “interphase cytoplasmic” or “IS15” extract. This cell extract may be aliquoted into 20 μl volumes of extract per tube on ice and immediately flash-frozen on liquid nitrogen and stored at −80° C. until use. Alternatively, the cell extract is placed in an ultracentrifuge tube on ice (e.g., fitted for an SW55 Ti rotor; Beckman). If necessary, the tube is overlayed with mineral oil to the top. The extract is centrifuged at 200,000×g for three hours at 4° C. to sediment membrane vesicles contained in the IS15 extract. At the end of centrifugation, the oil is discarded. The supernatant is carefully collected, pooled if necessary, and placed in a cold 1.5 ml tube on ice. This supernatant is referred to as “IS200” or “interphase cytosolic” extract. The extract is aliquoted and frozen as described for the IS15 extract. If desired, the extract can be enriched with additional nuclear factors. For example, nuclei can be purified from cells of the cell type from which the reprogramming extract is derived and lysed by sonication as described above. The nuclear factors are extracted by a 10-60 minute incubation in nuclear buffer containing NaCl or KCl at a concentration of 0.15-800 mM under agitation. The lysate is centrifuged to sediment unextractable components. The supernatant containing the extracted factors of interest is dialyzed to eliminate the NaCl or KCl. The dialyzed nuclear extract is aliquoted and stored frozen. This nuclear extract is added at various concentrations to the whole cell extract described above prior to adding the nuclei for reprogramming. As an alternative to a cell extract, a reprogramming media can also be formed by adding one or more naturally-occurring or recombinant factors (e.g., nucleic acids or proteins such as T-cell receptors or other signaling surface molecules, DNA methyltransferases, histone deacetylases, histones, nuclear lamins, transcription factors, activators, repressors, growth factors, hormones, or cytokines) to a solution, such as a buffer. Preferably, one or more of the factors are specific for the cell type one wishes the donor cell to become.

The extract can be used for reprogramming of fibroblasts by culture. In one embodiment, fibroblasts grown on coverslips are reversibly permeabilized with the bacterial toxin Streptolysin O, exposed to extracts of pluripotent stem cells and resealed with 2 mM CaCl.sub.2, and expanded in culture. In one embodiment, fibroblasts are grown on 16-mm poly-L-lysine-coated coverslips in RPMI1640 to 100,000 cells/coverslip in 12-well plates. Cells are permeabilized in 200 ng/ml streptolysin O in Ca²⁺-free Hanks Balanced Salt Solution (Gibco-BRL) for 50 minutes at 37° C. in regular atmosphere. Over 80% of fibroblasts cells are permeabilized under these conditions, as judged by propidium iodide uptake. Streptolysin O is aspirated; coverslips overlaid with 80 .mu.l of either pluripotent stem cell extract; and incubated for one hour at 37° C. in CO₂ atmosphere. Each extract contained the ATP generating system and 1 mM each of ATP, CTP, GTP and UTP. Extracts from pluripotent stem cells are prepared as described above. To reseal plasma membranes, RPMI1640 containing 2 mM CaCl₂) (added from a 1 M stock in H₂O) is added to the wells, and the cells are incubated for two hours at 37° C. This procedure resealed .about.100% of the permeabilized cells. Ca²⁺-containing RPMI was replaced by RPMI, and the cells are expanded for several weeks.

Several descriptions of cytoplasmic transferring have been published and are incorporated by reference [27-29].

### EXAMPLES

The following example is included to demonstrate particular embodiments of the disclosure. It should be appreciated by those of skill in the art that the techniques disclosed in the example that follows represent techniques discovered by the inventors to function well in the practice of the disclosed methods and compositions, and thus can be considered to constitute particular modes for its practice. However, those of skill in the art should, in light of the present disclosure, appreciate that many changes can be made in the specific embodiments that are disclosed and still obtain a like or similar result without departing from the spirit and scope of the disclosure.

### Example 1

### Fibroblasts Enhance Viability of Liver Cells in 3 Dimensional Culture

Foreskin fibroblasts (ATCC) and HepG2 hepatocytes were cultured at 37° C. in a 5% CO² incubator in DMEM with low glucose (Gibco), with supplement of 10% Fetal Bovine Serum (Gibco), 1% penicillin streptomycin (Gibco) and 1% glutar-max (Gibco). The growth medium was replaced every 3-4 days. At around 80% confluence, cells were isolated by trypsinization with 0.05% trypsin-EDTA (1×) (Gibco) briefly before re-suspending in full medium for subsequent experiments. Cells at P6 were used for subsequent experiments.

Subsequent to trypsinization cells were cultured to sub-confluence and were then detached by treating with 0.25% and 0.05% trypsin-EDTA(1×) (Gibco) for 5 minutes. Fibroblasts and HepG2 cells where mixed at 2:1 and 1:1 ratio. Rat tail type I collagen (Becton Dickenson Biosciences, Bedford, Mass.) was neutralized by 0.1N NaOH. Cell mixtures were suspended in neutralized collagen solution to make up cell-matrix mixtures with different final cell densities (2.5×10⁵, 5×10⁵ cells/ml and 1×10⁶ cells/ml, equivalent to 1250, 2500 and 5000 cells/5 μl droplet, respectively). Liquid droplets of cell-matrix mixtures were dispensed onto a non-adhesive surface, which is UV-irradiated parafilm in a 90-mm diameter Petri dish (Sterilin, London, United Kingdom), and then incubated at 37° C. with 5% CO² for 45 minutes to induce gelation. Gelated collagen microspheres containing both cells were gently flushed with a co-culture medium into a Petri dish for free-floating suspension cultures for different duration (24, 48 and 72 hours).

Viability of hepatocytes was confirmed by flow cytometry as follows: Single cell suspensions (1×10⁶ cells) obtained from collagenase-trypsin digestion of the microspheres were re-suspended in 500 of co-culture medium, incubated at room temperature for an hour to allow the recovery of cell surface protein expression, and were then fixed by 0.01% PFA for 15 minutes. Cells were then blocked by 2% Goat serum (Vector Laboratories) in PBS for 30 minutes before indirect staining of antibodies. To each sample, 1 μl of mouse monoclonal antibody against HepG2 cells (NB84a, abcam) in 2% Goat serum (dilution 1:100) was added. Isotype controls (normal mouse IgG antibody, Millipore) were performed at each time point. After staining at room temperature for 30 minutes, 1 ml PBS was added to each tube to wash off the excess antibodies. After centrifugation at 2000 rpm for 5 min, the supernatant was removed and 0.5 μl of Alexa Fluor 647 goat Anti-mouse secondary antibody (Invitrogen) in 2% goat serum (dilution 1:200) was added to each sample. After staining in dark at room temperature for 30 minutes, 1 ml PBS was added to each tube to wash off the excess antibodies. After centrifugation at 2000 rpm for 5 min, the supernatant was removed and Cell pellets were resuspended and preserved in 500 μl 1% PFA at a cell density not less than 4×10⁵ cells/ml for flow cytometry analysis in FACSCanto II Flow Cytometer (BD Biosciences, Bedford, Mass.). 10,000 events of each sample were analyzed. Results were analyzed with Flowing Software 2.5. Apoptosis of HepG2 was measured with Annexin V staining.

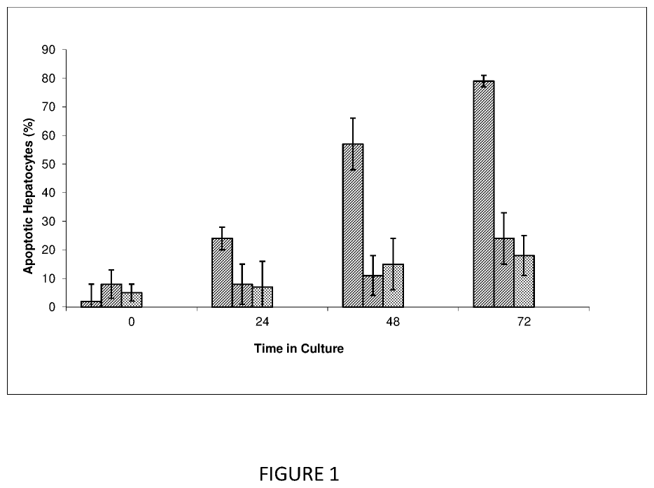

As seen in FIG. 1, enhanced viability was observed in the coculture with fibroblasts.

### REFERENCES

1. Vrana, K. E., et al., Nonhuman primate parthenogenetic stem cells. Proc Natl Acad Sci USA, 2003. 100 Suppl 1: p. 11911-6.
2. Sanchez-Pernaute, R., et al., Long-term survival of dopamine neurons derivedfrom parthenogenetic primate embryonic stem cells (cyno-1) after transplantation. Stem Cells, 2005. 23(7): p. 914-22.
3. Cibelli, J. B., K. Cunniff, and K. E. Vrana, Embryonic stem cellsfrom parthenotes. Methods Enzymol, 2006. 418: p. 117-35.
4. Revazova, E. S., et al., Patient-specific stem cell lines derived from human parthenogenetic blastocysts. Cloning Stem Cells, 2007. 9(3): p. 432-49.
5. de Fried, E. P., et al., Human parthenogenetic blastocysts derived from noninseminated cryopreserved human oocytes. Fertil Steril, 2008. 89(4): p. 943-7.
6. French, A. J., S. H. Wood, and A. O. Trounson, Human therapeutic cloning (NTSC): applying research from mammalian reproductive cloning. Stem Cell Rev, 2006. 2(4): p. 265-76.
7. Lin, G., et al., A highly homozygous and parthenogenetic human embryonic stem cell line derived from a one-pronuclear oocyte following in vitro fertilization procedure. Cell Res, 2007. 17(12): p. 999-1007.
8. Revazova, E. S., et al., HLA homozygous stem cell lines derived from human parthenogenetic blastocysts. Cloning Stem Cells, 2008. 10(1): p. 11-24.
9. De Sousa, P. A. and I. Wilmut, Human parthenogenetic embryo stem cells: appreciating what you have when you have it. Cell Stem Cell, 2007. 1(3): p. 243-4.
10. Wun, I. C. and R. E. Dittman, Human somatic cell nuclear transfer. Chin J Physiol, 2008. 51(4): p. 208-13.
11. Taupin, P., Parthenogenetically activated human oocytes and parthenogenetic embryonic stem cells: US20100233143. Expert Opin Ther Pat, 2011. 21(8): p. 1281-3.
12. Wei, Q., et al., Derivation of rhesus monkey parthenogenetic embryonic stem cells and its microRNA signature. PLoS One, 2011. 6(9): p. e25052.
13. Yabuuchi, A., H. Rehman, and K. Kim, Histocompatible parthenogenetic embryonic stem cells as a potential source for regenerative medicine. J Mamm Ova Res, 2012. 29(1): p. 17-21.
14. Daughtry, B. and S. Mitalipov, Concise review: parthenote stem cellsfor regenerative medicine: genetic, epigenetic, and developmental features. Stem Cells Transl Med, 2014. 3(3): p. 290-8.
15. Espej el, S., et al., Brief report: Parthenogenetic embryonic stem cells are an effective cell sourcefor therapeutic liver repopulation. Stem Cells, 2014. 32(7): p. 1983-8.
16. Cervera, R. P. and M. Stojkovic, Human embryonic stem cell derivation and nuclear transfer: impact on regenerative therapeutics and drug discovery. Clin Pharmacol Ther, 2007. 82(3): p. 310-5.
17. De Sousa, P. A., et al., Clinically failed eggs as a source of normal human embryo stem cells. Stem Cell Res, 2009. 2(3): p. 188-97.
18. Takahashi, K. and S. Yamanaka, Induction of pluripotent stem cells from mouse embryonic and adult fibroblast cultures by defined factors. Cell, 2006. 126(4): p. 663-76.
19. Park, I. H., et al., Reprogramming of human somatic cells to pluripotency with defined factors. Nature, 2008. 451(7175): p. 141-6.
20. Chhabra, A., Derivation of Human Induced Pluripotent Stem Cell (iPSC) Lines and Mechanism of Pluripotency: Historical Perspective and Recent Advances. Stem Cell Rev, 2017.
21. Shi, Y., et al., Induced pluripotent stem cell technology: a decade of progress. Nat Rev Drug Discov, 2017. 16(2): p. 115-130.
22. Kele, M., et al., Generation of human iPS cell line CTL07-II from human fibroblasts, under defined and xeno-free conditions. Stem Cell Res, 2016. 17(3): p. 474-478.
23. Obokata, H., et al., Bidirectional developmental potential in reprogrammed cells with acquired pluripotency. Nature, 2014. 505(7485): p. 676-80.
24. Zhou, Q., et al., A comparative approach to somatic cell nuclear transfer in the rhesus monkey. Hum Reprod, 2006. 21(10): p. 2564-71.
25. Hall, V. J., et al., Developmental competence ofhuman in vitro aged oocytes as host cellsfor nuclear transfer. Hum Reprod, 2007. 22(1): p. 52-62.
26. Sung, L. Y., et al., Efficient derivation of embryonic stem cells from nuclear transfer and parthenogenetic embryos derived from cryopreserved oocytes. Cell Reprogram, 2010. 12(2): p. 203-11.
27. Collas, P. and C. K. Taranger, Epigenetic reprogramming ofnuclei using cell extracts. Stem Cell Rev, 2006. 2(4): p. 309-17.
28. Collas, P. and C. K. Taranger, Toward reprogramming cells to pluripotency. Ernst Schering Res Found Workshop, 2006(60): p. 47-67.
29. Collas, P., et al., On the way to reprogramming cells to pluripotency using cell-free extracts. Reprod Biomed Online, 2006. 12(6): p. 762-70.

Although the present disclosure and its advantages have been described in detail, it should be understood that various changes, substitutions and alterations can be made herein without departing from the spirit and scope of the design as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the present disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

What is claimed is:

1. A method of generating an organ, organoid, and/or tissue, comprising the steps of:
a. culturing fibroblasts under suitable conditions to induce dedifferentiation of fibroblasts; and
b. using said dedifferentiated fibroblasts, or differentiated cells produced therefrom, in a bio-printing process to produce the organ, organoid and/or tissue.

2. The method of claim 1, wherein said fibroblasts are selected from the group consisting of dermal fibroblasts, placental fibroblasts, adipose fibroblasts, bone marrow fibroblasts, foreskin fibroblasts, umbilical cord fibroblasts, hair follicle-derived fibroblasts, nail-derived fibroblasts, endometrial derived fibroblasts, keloid derived fibroblasts, and a combination thereof.

3. The method of claim 1 or 2, wherein in said culturing step said fibroblasts are subjected to hypoxic conditions.

4. The method of claim 3, wherein said hypoxic conditions comprise a reduced oxygen range between 0.2%-5%.

5. The method of any one of claims 1-4, wherein said culturing step comprises culturing of the fibroblasts in a culture medium treated with one or more epigenetic modifiers.

6. The method of claim 5, wherein said epigenetic modifier is a DNA demethylating agent, histone deacetylase inhibitor, histone modifier, or a combination thereof.

7. The method of claim 6, wherein said DNA demethylating agent is selected from the group consisting of 5-azacytidine, N-methyl-N′-nitro-N-nitrosoguanidine (MNNG), Temozolomide, Procarbazine, and a combination thereof.

8. The method of claim 6, wherein said histone deacetylase inhibitor is selected from the group consisting of Valproic acid, Trichostatin A, Phenylbutyrate, Vorinostat, Belinostat, LAQ824, Panobinostat, Entinostat, CI994, Mocetinostat, Sulforaphane, and a combination thereof.

9. The method of claim 6 wherein said histone modifier is selected from the group consisting of poly ADP ribose polymerase, Enhancer of zeste homolog 2 (EZH2), valproic acid, trichostatine, and a combination thereof.

10. The method of any one of claims 1-9, wherein said fibroblasts are transfected with cytoplasm derived from stem cells.

11. The method of claim 10, wherein said stem cells are selected from the group consisting of parthenogenic stem cells, embryonic stem cells, inducible pluripotent stem cells, somatic cell nuclear transfer derived stem cells, and a combination thereof.

12. The method of claim 10, wherein said fibroblasts transfected with cytoplasm derived from pluripotent stem cells become dedifferentiated.

13. The method of claim 12, wherein said dedifferentiated fibroblasts express detectable levels of one or more genes selected from the group consisting of alkaline phosphatase (ALP), OCT4, SOX2, human telomerase reverse transcriptase (hERT), SSEA-4, NANOG, and a combination thereof.

14. The method of claim 12, wherein said dedifferentiated fibroblasts are cultured using a culture medium treated with one or more growth factors.

15. The method of claim 14, wherein said growth factors are selected from the group consisting of FGF-1, FGF-2, FGF-5, EGF, CNTF, KGF-1, PDGF, platelet-rich plasma, TGF-alpha, HGF-1, and a combination thereof.

16. The method of any one of claims 1-15, wherein the fibroblasts, dedifferentiated fibroblasts, or differentiated cells produced therefrom, are cultured as a plurality of cell aggregates.

17. The method of claim 16, wherein the fibroblasts, dedifferentiated fibroblasts, or differentiated cells produced therefrom, are cultured as a plurality of cell aggregates prior to the bioprinting process.

18. The method of claim 16 or 17, wherein said cell aggregates are seeded in extracellular matrix.

19. The method of claim 18, wherein said extracellular matrix is selected from the group consisting of mammalian extracellular matrix, piscine extracellular matrix, plant extracellular matrix, and a combination thereof.

20. The method of any one of claims 1-19, further comprising the steps of generating extracellular matrix from fibroblast lysates.

21. The method of claim 18, wherein said cell aggregates are seeded in extracellular matrix that is configured into the form of a defined shape.

22. The method of claim 21, wherein said cell aggregates seeded in extracellular matrix are introduced into a mold having the defined shape.

23. The method of any one of claims 1-22, wherein said fibroblasts, dedifferentiated fibroblasts, or differentiated cells produced therefrom are subjected to hypoxic conditions.

24. The method of claim 18, wherein said extracellular matrix is cultured using a culture medium treated with one or more cell attachment peptides, one or more cell attachment proteins, one or more cytokines, one or more glycosaminoglycans, or a combination thereof.

25. The method of claim 24, wherein said cell attachment peptide is RGD peptide

26. The method of claim 24, wherein said cell attachment protein is RGD peptide

27. The method of claim 24, wherein said cytokine is selected from the group consisting of vascular endothelial growth factor (VEGF), bone morphogenetic protein (BMP), adrenomedullin (AM), angiopoietin (Ang), brain-derived neurotrophic factor (BDNF), epidermal growth factor (EGF), erythropoietin (Epo), fibroblast growth factor (FGF), glial cell line-derived neurotrophic factor (GNDF), granulocyte colony stimulating factor (G-CSF), granulocyte-macrophage colony stimulating factor (GM-CSF), growth differentiation factor (GDF-9), hepatocyte growth factor (HGF), hepatoma-derived growth factor (HDGF), insulin-like growth factor (IGF), migration-stimulating factor, myostatin (GDF-8), myelomonocytic growth factor (MGF), nerve growth factor (NGF), placental growth factor (P1GF), platelet-derived growth factor (PDGF), thrombopoietin (Tpo), transforming growth factor alpha (TGFα), transforming growth factor beta (TGFβ), tumor necrosis factor alpha (TNFα), Wnt protein, and a combination thereof.

28. The method of claim 24, wherein said glycosaminoglycan is selected from the group consisting of hyaluronate, chondroitin sulfate, heparin sulfate, heparin, dermatan sulfate, and keratin sulfate, and a combination thereof.

29. The method of claim 18, further comprising admixing a hydrogel during deposition of the matrix onto said cells or cellular aggregates.

30. The method of claim 18, further comprising deposition of a synthetic polymer onto the cells and/or cellular aggregates alone, or that have been admixed with a hydrogel.

31. The method of claim 30, wherein said synthetic polymer is selected from the group consisting of poly (L-lactide-co-glycolide), poly lactic-co-glycolic acid (PLGA), Polycaprolactone (PLC), Polylactic acid, Polybutylene terephthalate, Polyethylene terephthalate, Polyethylene glycol, and a combination thereof.

32. The method of any one of claims 1-31, wherein dedifferentiated fibroblasts and/or differentiated cells produced therefrom are of an endodermal, ectodermal, or mesodermal lineage.

33. The method of any one of claims 1-32, wherein said dedifferentiated fibroblasts are differentiated into cells of a desired type.

34. The method of claim 33, wherein said dedifferentiated fibroblasts are differentiated into cells of a desired type selected from the group consisting of salivary gland mucous cells, salivary gland serous cells, von Ebner's gland cells, mammary gland cells, lacrimal gland cells, ceruminous gland cells, eccrine sweat gland dark cells, eccrine sweat gland clear cells, apocrine sweat gland cells, gland of Moll cells, sebaceous gland cells, Bowman's gland cells, Brunner's gland cells, seminal vesicle cells, prostate gland cells, bulbourethral gland cells, Bartholin's gland cells, Littre gland cells, uterus endometrium cells, goblet cells, stomach lining mucous cells, gastric gland zymogenic cells, gastric gland oxyntic cells, pancreatic acinar cells, paneth cells, type II pneumocytes, clara cells, somatotropes, lactotropes, thyrotropes, gonadotropes, corticotropes, intermediate pituitary cells, magnocellular neurosecretory cells, gut cells, respiratory tract cells, thyroid epithelial cells, parafollicular cells, parathyroid gland cells, parathyroid chief cells, oxyphil cells, adrenal gland cells, chromaffin cells, Leydig cells, theca interna cells, corpus luteum cells, granulosa lutein cells, theca lutein cells, juxtaglomerular cells, macula densa cells, peripolar cells, mesangial cells, blood vessel and lymphatic vascular endothelial fenestrated cells, blood vessel and lymphatic vascular endothelial continuous cells, blood vessel and lymphatic vascular endothelial splenic cells, synovial cells, peritoneal serosal cells, pleural serosal cells, pericardial cavity serosal cells, squamous cells, columnar cells, dark cells, vestibular membrane cells, stria vascularis basal cells, stria vascularis marginal cells, cells of Claudius, cells of Boettcher, choroid plexus cells, arachnoid squamous cells, pigmented ciliary epithelium cells, non-pigmented ciliary epithelium cells, corneal endothelial cells, peg cells, respiratory tract ciliated cells, oviduct ciliated cells, uterine endometrial ciliated cells, rete testis ciliated cells, ductulus efferens ciliated cells, ciliated ependymal cells, epidermal keratinocytes, epidermal basal cells, fingernail and toenail keratinocytes, nail bed basal cells, medullary hair shaft cells, cortical hair shaft cells, cuticular hair shaft cells, cuticular hair root sheath cells, hair root sheath cells of Huxley's layer, hair root sheath cells of Henle's layer, external hair root sheath cells, hair matrix cells, stratified squamous epithelium, epithelial basal cells, urinary epithelium cells, inner auditory hair cells of the organ of Corti, outer auditory hair cells of the organ of Corti, basal cells of olfactory epithelium, cold-sensitive primary sensory neurons, heat-sensitive primary sensory neurons, epidermal Merkel cells, olfactory receptor neurons, pain-sensitive primary sensory neurons, photoreceptor rod cells, photoreceptor blue-sensitive cone cells, photoreceptor green-sensitive cone cells, photoreceptor red-sensitive cone cells, proprioceptive primary sensory neurons, touch-sensitive primary sensory neurons, type I carotid body cells, type II carotid body cells, type I hair cell of the vestibular apparatus of the ear, type II hair cell of the vestibular apparatus of the ear, type I taste bud cells, cholinergic neural cells, adrenergic neural cells, peptidergic neural cells, inner pillar cells of the organ of Corti, outer pillar cells of the organ of Corti, inner phalangeal cells of the organ of Corti, outer phalangeal cells of the organ of Corti, border cells of the organ of Corti, Hensen cells of the organ of Corti, vestibular apparatus supporting cells, taste bud supporting cells, olfactory epithelium supporting cells, Schwann cells, satellite cells, enteric glial cells, astrocytes, neurons, oligodendrocytes, spindle neurons, anterior lens epithelial cells, crystallin-containing lens fiber cells, hepatocytes, adipocytes, white fat cells, brown fat cells, liver lipocytes, kidney glomerulus parietal cells, kidney glomerulus podocytes, kidney proximal tubule brush border cells, loop of Henle thin segment cells, kidney distal tube cells, kidney collecting duct cells, type I pneumocytes, pancreatic duct cells, non-striated duct cells, duct cells, intestinal brush border cells, exocrine gland striated duct cells, gall bladder epithelial cells, ductus efferens non-ciliated cells, epididymal principal cells, epididymal basal cells, ameloblast epithelial cells, planum semilunatum epithelial cells, organ of Corti interdental epithelial cells, loose connective tissue fibroblasts, corneal keratocytes, tendon fibroblasts, bone marrow reticular tissue fibroblasts, non-epithelial fibroblasts, pericytes, nucleus pulposus cells, cementoblast/cementocytes, odontoblasts, odontocytes, hyaline cartilage chondrocytes, fibrocartilage chondrocytes, elastic cartilage chondrocytes, osteoblasts, osteocytes, osteoclasts, osteoprogenitor cells, hyalocytes, cochlear stellate cells, hepatic stellate cells, pancreatic stellate cells, red skeletal muscle cells, white skeletal muscle cells, intermediate skeletal muscle cells, nuclear bag cells of the muscle spindle, nuclear chain cells of the muscle spindle, satellite cells, cardiomyocytes, nodal cardiomyocytes, Purkinje fiber cells, smooth muscle cells, myoepithelial cells of the iris, myoepithelial cells of the exocrine glands, reticulocytes, megakaryocytes, monocytes, connective tissue macrophages, epidermal Langerhans cells, dendritic cells, microglial cells, neutrophils, eosinophils, basophils, mast cells, helper T cells, suppressor T cells, cytotoxic T cells, natural killer T cells, B cells, natural killer cells, melanocytes, retinal pigmented epithelial cells, oogonia/oocytes, spermatids, spermatocytes, spermatogonium cells, spermatozoa, ovarian follicle cells, Sertoli cells, thymus epithelial cells, and intestinal kidney cells.

35. The method of any one of claims 1-34, wherein bioprinting comprises three-dimensional printing of a biological organ, organoid, and/or tissue through the layering of living cells using a bioprinter.

36. The method of claim 35, wherein said bioprinter is a three-axis mechanical platform that controls the movements of extruders that deposit layers of living cells in a desired shape.

37. The method of claim 36, wherein said desired shape is acquired by scanning the surface of a desired organ, organoid and/or tissue to generate a surface map for guidance with cell deposition.

38. The method of claim 37, wherein scanning the surface of a desired organ, organoid and/or tissue is achieved using a laser, electron beam, magnetic resonance imaging, microwave, x-ray, computed tomography, or a combination thereof.

39. A method of preparing cells for a bioprinting process, comprising the step of exposing fibroblasts to suitable conditions to cause dedifferentiation of the fibroblasts.

40. The method of claim 39, wherein the dedifferentiated fibroblasts are subject to suitable conditions to differentiate into differentiated cells of a desired type.

41. The method of claim 40, wherein the differentiated cells of a desired type are selected from the group consisting of salivary gland mucous cells, salivary gland serous cells, von Ebner's gland cells, mammary gland cells, lacrimal gland cells, ceruminous gland cells, eccrine sweat gland dark cells, eccrine sweat gland clear cells, apocrine sweat gland cells, gland of Moll cells, sebaceous gland cells, Bowman's gland cells, Brunner's gland cells, seminal vesicle cells, prostate gland cells, bulbourethral gland cells, Bartholin's gland cells, Littre gland cells, uterus endometrium cells, goblet cells, stomach lining mucous cells, gastric gland zymogenic cells, gastric gland oxyntic cells, pancreatic acinar cells, paneth cells, type II pneumocytes, clara cells, somatotropes, lactotropes, thyrotropes, gonadotropes, corticotropes, intermediate pituitary cells, magnocellular neurosecretory cells, gut cells, respiratory tract cells, thyroid epithelial cells, parafollicular cells, parathyroid gland cells, parathyroid chief cells, oxyphil cells, adrenal gland cells, chromaffin cells, Leydig cells, theca interna cells, corpus luteum cells, granulosa lutein cells, theca lutein cells, juxtaglomerular cells, macula densa cells, peripolar cells, mesangial cells, blood vessel and lymphatic vascular endothelial fenestrated cells, blood vessel and lymphatic vascular endothelial continuous cells, blood vessel and lymphatic vascular endothelial splenic cells, synovial cells, peritoneal serosal cells, pleural serosal cells, pericardial cavity serosal cells, squamous cells, columnar cells, dark cells, vestibular membrane cells, stria vascularis basal cells, stria vascularis marginal cells, cells of Claudius, cells of Boettcher, choroid plexus cells, arachnoid squamous cells, pigmented ciliary epithelium cells, non-pigmented ciliary epithelium cells, corneal endothelial cells, peg cells, respiratory tract ciliated cells, oviduct ciliated cells, uterine endometrial ciliated cells, rete testis ciliated cells, ductulus efferens ciliated cells, ciliated ependymal cells, epidermal keratinocytes, epidermal basal cells, fingernail and toenail keratinocytes, nail bed basal cells, medullary hair shaft cells, cortical hair shaft cells, cuticular hair shaft cells, cuticular hair root sheath cells, hair root sheath cells of Huxley's layer, hair root sheath cells of Henle's layer, external hair root sheath cells, hair matrix cells, stratified squamous epithelium, epithelial basal cells, urinary epithelium cells, inner auditory hair cells of the organ of Corti, outer auditory hair cells of the organ of Corti, basal cells of olfactory epithelium, cold-sensitive primary sensory neurons, heat-sensitive primary sensory neurons, epidermal Merkel cells, olfactory receptor neurons, pain-sensitive primary sensory neurons, photoreceptor rod cells, photoreceptor blue-sensitive cone cells, photoreceptor green-sensitive cone cells, photoreceptor red-sensitive cone cells, proprioceptive primary sensory neurons, touch-sensitive primary sensory neurons, type I carotid body cells, type II carotid body cells, type I hair cell of the vestibular apparatus of the ear, type II hair cell of the vestibular apparatus of the ear, type I taste bud cells, cholinergic neural cells, adrenergic neural cells, peptidergic neural cells, inner pillar cells of the organ of Corti, outer pillar cells of the organ of Corti, inner phalangeal cells of the organ of Corti, outer phalangeal cells of the organ of Corti, border cells of the organ of Corti, Hensen cells of the organ of Corti, vestibular apparatus supporting cells, taste bud supporting cells, olfactory epithelium supporting cells, Schwann cells, satellite cells, enteric glial cells, astrocytes, neurons, oligodendrocytes, spindle neurons, anterior lens epithelial cells, crystallin-containing lens fiber cells, hepatocytes, adipocytes, white fat cells, brown fat cells, liver lipocytes, kidney glomerulus parietal cells, kidney glomerulus podocytes, kidney proximal tubule brush border cells, loop of Henle thin segment cells, kidney distal tube cells, kidney collecting duct cells, type I pneumocytes, pancreatic duct cells, non-striated duct cells, duct cells, intestinal brush border cells, exocrine gland striated duct cells, gall bladder epithelial cells, ductus efferens non-ciliated cells, epididymal principal cells, epididymal basal cells, ameloblast epithelial cells, planum semilunatum epithelial cells, organ of Corti interdental epithelial cells, loose connective tissue fibroblasts, corneal keratocytes, tendon fibroblasts, bone marrow reticular tissue fibroblasts, non-epithelial fibroblasts, pericytes, nucleus pulposus cells, cementoblast/cementocytes, odontoblasts, odontocytes, hyaline cartilage chondrocytes, fibrocartilage chondrocytes, elastic cartilage chondrocytes, osteoblasts, osteocytes, osteoclasts, osteoprogenitor cells, hyalocytes, cochlear stellate cells, hepatic stellate cells, pancreatic stellate cells, red skeletal muscle cells, white skeletal muscle cells, intermediate skeletal muscle cells, nuclear bag cells of the muscle spindle, nuclear chain cells of the muscle spindle, satellite cells, cardiomyocytes, nodal cardiomyocytes, Purkinje fiber cells, smooth muscle cells, myoepithelial cells of the iris, myoepithelial cells of the exocrine glands, reticulocytes, megakaryocytes, monocytes, connective tissue macrophages, epidermal Langerhans cells, dendritic cells, microglial cells, neutrophils, eosinophils, basophils, mast cells, helper T cells, suppressor T cells, cytotoxic T cells, natural killer T cells, B cells, natural killer cells, melanocytes, retinal pigmented epithelial cells, oogonia/oocytes, spermatids, spermatocytes, spermatogonium cells, spermatozoa, ovarian follicle cells, Sertoli cells, thymus epithelial cells, and intestinal kidney cells.

42. The method of claim 40 or 41, comprising the step of employing the differentiated cells of a desired type in a bioprinting process.
